(19) United States
(12) Patent Application Publication
(10) Pub. No.: US 2025/0332090 A1
(43) Pub. Date: Oct. 30, 2025

(54) TOPICAL FORMULATION COMPRISING OMEGA-3 FATTY ACIDS, MELATONIN AND VITAMIN D

(71) Applicant: Ambo Innovations LLC, Osprey, FL (US)

(72) Inventor: Bo Martinsen, Osprey, FL (US)

(21) Appl. No.: 18/864,389

(22) PCT Filed: May 11, 2023
(86) PCT No.: PCT/US23/21888

§ 371 Date: Nov. 8, 2024

Related U.S. Application Data
(60) Provisional application No. 63/340,663, filed on May 11, 2022.

(51) Int. Cl. A61K 9/00; A61K 9/06; A61K 31/00; A61K 31/07; A61K 31/192; A61K 31/202; A61K 31/4045; A61K 31/593; A61K 31/704; A61K 31/716; A61K 35/60; A61K 36/236; A61K 36/30; A61K 36/61; A61K 36/889; A61K 45/06; A61K 47/10; A61K 47/20; A61K 47/24; A61K 47/36; A61P 17/02; A61P 17/08; A61P 25/20; A61P 27/04

(52) U.S. Cl. CPC A61K 9/0014; A61K 9/0043; A61K 9/006; A61K 9/06; A61K 31/07; A61K 31/192; A61K 31/202; A61K 31/4045; A61K 31/593; A61K 31/658; A61K 31/704; A61K 31/716; A61K 35/60; A61K 36/236; A61K 36/30; A61K 36/61; A61K 36/889; A61K 45/06; A61K 47/10; A61K 47/20; A61K 47/24; A61K 47/36; A61P 17/02; A61P 17/08; A61P 25/20; A61P 27/04

(57) ABSTRACT

The present disclosure provides topical formulations comprising omega-3 fatty acids, melatonin, vitamin D3, and β-glucan. The formulations are designed for application to either the skin or mucosa of a subject in need thereof. The present disclosure also provides methods of treatment or prevention of a variety of conditions by applying the formulations to the skin or mucosa of the subject, including various skin conditions, eye disorders and sleep disorders.

### CROSS-REFERENCE TO RELATED APPLICATIONS

This application claims the benefit of priority to U.S. Provisional application No. 63/340,663 filed on May 11, 2022, the contents of which are hereby incorporated by reference in their entirety.

### TECHNICAL FIELD

The present disclosure generally relates to topical formulations comprising omega-3 fatty acids, melatonin, and vitamin D. In some embodiments, the topical formulations further comprise β-glucan. The formulations also can serve as a delivery system for additional biologically active agents. The present disclosure also generally relates to methods of applying the topical formulations to the skin and/or mucosal membranes and serving as delivery system for additional cell nutrients or bioactive ingredients.

### BACKGROUND OF THE INVENTION

In the last few years there has been an emergence of a new understanding of pathology, based on the understanding that organ function is highly regulated or influenced by the integrity of surface barriers and their residing microbiome. An imbalance of the microbiota (dysbiosis) and poor barriers not only negatively influences cell metabolism but also reduces immune system aptness and even response to medications and medical treatments. With this understanding, three targets are evident when we consider prevention or treatment of diseases: 1) rebalancing dysbiosis, 2) strengthening surface barriers to prevent toxic substances, allergens, or pathogens from entering the body, and 3) reducing a dysregulated immune response to external stimuli typically referred to as chronic inflammation. Addressing these three targets at the same time would be more effective than just one at a time (anti-inflammation effects are the typical target of existing medications), as they constitute a continuous vicious cycle.

Even though the build-up of the different surface barriers are histologically different from site to site, they share the common feature of the lining epithelial cells being held together by so-called tight junctions. Epithelial barriers constitute dynamic structures that are influenced by a multitude of local cellular and extra cellular factors, which may not work properly and subsequently result in ineffective barriers and leak of toxic materials or pathogens through their domain into the body. More and more studies indicate that dysbiosis is part of a vicious cycle with ineffective barrier functions increasing risk of pathogens or toxic substances to pass through tight junction areas and setting off cellular immune response by activating inflammation receptors. Similar principals apply to the mucosal membranes, e.g., nasal or oral cavity membranes.

Lately, the nose mucosal membrane has received a lot of interest. SARS-Cov-2 and many other viruses can enter the nose to infect the body. Thus, strengthening these barriers may provide a method of preventing viral or microbial infections.

The nose has also received attention as an ideal site for administrating medications to affect the central nervous system because the molecules can be absorbed by the mucosa into the lymphatic tissue or cranial nerves and potentially pass the blood-brain barrier without being metabolized by the liver. The nose (like the ocular tissue) is also rich with epithelial-embedded transient receptor potential mediators (TRP), which communicates directly with the brain upon stimulation and constitute first line immune response to ambient stimuli.

Omega-3 fatty acids are essential to life and functioning of the cells and organs. They are important regulators of cell membrane lipid rafts and thereby influence cell signaling, communications and life cycles and serve as anchors for tight junction proteins and their regulation. Omega-3 fatty acids are especially found in fish oils. While fish and fish oil's positive health effects have been recognized for several thousand years and now attributed to their content of unsaturated fatty acids, the same substances have been dreaded because of low stability and their proneness to oxidation, resulting in bad smell and taste, which has limited their use in dermatology or on barrier surfaces. Additionally, formulating topical preparations to penetrate the top surface of the skin or reach lower viable layers of the epidermis is a challenge.

Accordingly, there is a need for formulations and methods for 1) rebalancing dysbiosis, 2) strengthening surface barriers to prevent toxic substances, allergens, or pathogens from entering the body, and 3) reducing a dysregulated immune response. There is further need of formulations and methods for treating and preventing skin cancers, chronic skin and mucosal inflammation disorders, and disturbed surfaces microbiome. There is also a strong need for finding new and more effective topical remedies that are well tolerated by the skin or mucosa and highly accepted by users. The present disclosure addresses these needs.

### SUMMARY

The present disclosure provides topical formulations comprising omega-3 fatty acids, melatonin, vitamin D3 and optionally β-glucan as set forth in the following non-limiting embodiments.

In some embodiments, the topical formulation comprises:
an omega-3 fatty acid component comprising eicosapentaenoic acid (EPA), docosahexaenoic acid (DHA), or a combination thereof;
melatonin;
vitamin D3;
β-glucan; and
at least one topical carrier.

In some embodiments, the topical formulation of claim 1, wherein the omega-3 fatty acid comprises EPA and DPA. The EPA and DHA may have a EPA:DHA weight ratio ranging from about 0.5:1.5 to about 1.5:0.5. In some embodiments, the EPA:DHA weight ratio ranges from about 0.75:1.25 to about 1.25:0.75; about 0.8:1.2 to about 1.2:0.8; or about 0.9:1.1 to about 1.1:0.9, while in further embodiments, the EPA:DHA weight ratio is about 1:1. In still further embodiments, the omega-3 fatty acid consists essentially of the EPA and the DHA.

In some embodiments, the omega-3 fatty source is purified fish oil. In some embodiments, the purified fish oil is purified fish liver oil.

In some embodiments, the topical formulation further comprises a source of GLA, such as borage oil.

In some embodiments, the topical formulation further comprises eucalyptus oil. In some embodiments, the topical formulation comprises about 0.1 to about 5% by weight of the eucalyptus oil, or 0.1 to about 1% by weight of the eucalyptus oil.

Topical formulations disclosed herein may further comprise at least one cannabinoid. The cannabinoid may be chosen from cannabidiol (CBD), cannabidiolic acid (CBDA), cannabinol (CBN), Cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), and any combination thereof.

In some embodiments, the topical formulation comprises formulated as a gel, lotion, solution, cream, ointment, oil, dressing, foam, spray, or film, and in particular embodiments, a cream, ointment, or lotion.

In some embodiments, the topical carrier comprises water, hyaluronic acid, lecithin, DMSPO and/or glycerin. In some embodiments, the topical formulation comprises about 60 to about 85% by weight of the water.

In some embodiments, the topical formulation is formulated for application to skin. In some embodiments, the formulation is formulated for application to mucosa. In some embodiments, the formulation is formulated for application to the eye lids.

Topical formulations disclosed herein may further comprise at least one additional biologically active agent, such as an antibiotic, an anti-inflammatory, an anti-cancer, an enzyme inhibitor, a TRP modulator, a topical steroid, or an analgesic.

The present disclosure further provides methods or treatment and prevention of various conditions using the present formulations. In some embodiments, a method for treating or preventing dysbiosis and/or strengthening surface barriers of the skin or mucosa of a subject in need thereof comprises applying to the skin or mucosa an effective amount of a topical formulation described herein. In some embodiments, the mucosa is nasal, oral, ocular, genital, anal, or rectal. In some embodiments, the nasal mucosa or oral mucosa., while in particular embodiments, the mucosa is nasal mucosa.

In some embodiments, a method for promoting sleep or treating or preventing a sleep disorder in a subject in need thereof comprises applying the topical formulation described herein to the eye lids or nasal mucosa of the subject. The sleep disorder may be sleep apnea, jet lag, or insomnia.

In some embodiments, a method for treating or preventing a mucosal site condition in a subject in need thereof comprises applying an effective amount the topical formulation described herein to the mucosa of the subject. In some embodiments, the mucosa is nasal, oral, ocular, genital, anal, or rectal mucosa, and in more particular embodiments, the mucosa is nasal mucosa.

In some embodiments, the mucosal site condition is pollen allergy, chronic rhinosinusitis, nasal dysbiosis, asthma, viral infection, bacterial infection, or any combination thereof. In some embodiments, the method comprises applying the formulation by nasal spray, swab, or squeeze tube. In some embodiments, the effective amount of the topical formulation is about 50 mg to about 200 mg. of the formulation. In some embodiments, each 1 mL volume contains about 1 g (1000 mg) of the formulation.

In some embodiments, the mucosa is the oral mucosa. In these embodiments, the mucosal site condition is mucosal dryness, gingivitis, periodontitis, oral lichen planus, herpes labialis, candidiasis, ulcer, or any combination thereof.

In some embodiments, a method for treating or preventing a dermatological condition in a subject in need thereof comprises applying an effective amount of the formulation described herein to the skin of the subject. In some embodiments, the dermatological condition comprises eczema, pruritus, psoriasis, rash, hives, contact dermatitis, insect bite, allergic reaction, rosacea, acne, cold sores, blisters, hives, actinic keratosis, fungal infections, insect stings, burns, frost-bite, sun burn, UV radiation protection, post-surgical cuts, stomal site irritation, radiation therapy skin burns, photo-aging, abrasions, basal cell carcinoma, squamous cell carcinoma, melanoma, bed sores, parasitic infection, spider bites, shingles, infected wounds, seborrheic dermatitis, contact dermatitis, burns, post-surgical cuts or any combination thereof.

In some embodiments, a method for preventing or reducing dry eye, ocular tension, glaucoma, or retinal conditions in a subject in need thereof comprises applying a formulation described herein to the eye or eye lids of a subject in need thereof. In some embodiments, the method further comprises applying the formulation to the nasal mucosa.

In some embodiments, a method of treating vaginal dryness, vaginal or vulvar atrophy, or hot flashes comprises applying to the skin or mucosa an effective amount of a topical formulation described herein to the vaginal mucosa. In some embodiments, the disorder or condition is vaginal dryness, vaginal or vulvar atrophy.

In some embodiments, a perioperative treatment method for cataract or lasik surgery comprises applying a topical formulation described herein to the eye or eye lids of a subject in need thereof.

In some embodiments, a method of preventing or reducing ear ache in a subject in need thereof comprising applying a topical formulation described herein the ear canal of a subject in need thereof.

In some embodiments, a method of topically delivering a biologically active agent comprising applying an effective amount of a composition a topical formulation described herein to the skin or mucosa of a subject in need thereof.

In some embodiments of the above-described methods, the effective amount is about 0.5 mL to about 2 mL of the formulation.

### BRIEF DESCRIPTION OF THE DRAWINGS

FIG. 1A is a photograph of a chronic wound, and FIG. 1B is photograph of the wound site after three weeks of daily treatment with an exemplary composition of the invention.

FIG. 2A is a photograph of a papilloma, and FIG. 2B is photograph of the papilloma after 10 days of daily treatment with an exemplary composition of the invention.

FIG. 3A is a photograph of eczema of the foot and FIG. 3B is a photograph of the eczema site after two weeks of daily treatment with an exemplary composition of the invention.

FIG. 4A is a photograph of a cancer irradiation burn and FIG. 4B is a photograph of site of the irradiation burn after daily treatment for two and half weeks with an exemplary composition of the invention.

FIG. 5A is a photograph of a site of actinic keratosis and FIG. 5B is a photograph of site of actinic keratosis after daily treatment for five weeks with an exemplary composition of the invention.

FIG. 6A is a photograph of a site of chronic hand eczema and FIG. 6B is a photograph of site of the chronic hand eczema after daily treatment for two and a half weeks with an exemplary composition of the invention.

FIG. 7A is a photograph of a site periorbital wrinkles and FIG. 7B is a photograph of the site of periorbital wrinkles after daily treatment for six weeks with an exemplary composition of the invention.

FIG. 8 is a photograph of two sites of wasps stings after applying an exemplary formulation of the invention to the sites of the stings.

### DETAILED DESCRIPTION

The present disclosure provides, in some embodiments, topical formulations comprising an omega-3 fatty acid component, melatonin, vitamin D3, and a topical carrier. In some embodiments, the topical formulation comprises an omega-3 fatty acid component, melatonin, vitamin D3, β-glucan, and a topical carrier. The present topical formulations in some embodiments advantageously comprise ingredients that are either FDA or GRAS approved, and furthermore, may be naturally derived. Furthermore, it is believed that the ingredients synergistically affect the microbiome, surface barrier, and immune response, and are capable of strengthening skin and mucosal barriers. The formulations include a high omega-3 content, but are still rheologically stable, quickly absorbed, and do not have a strong odor (fishy smell) or staining. The topical formulations furthermore can serve as both a therapeutic agent itself, and as a carrier for topical delivery of other pharmaceutical agents.

All numeric ranges are inclusive of narrower ranges; delineated upper and lower range limits are interchangeable to create further ranges not explicitly delineated. The number of significant digits conveys neither limitation on the indicated amounts nor on the accuracy of the measurements.

In this document, the terms “a” or “an” are used to include one or more than one and the term “or” is used to refer to a nonexclusive “or” unless otherwise indicated. By way of example, “an element” means one element or more than one element.

The term “about,” as used herein, means approximately, in the region of, roughly, or around. When the term “about” is used in conjunction with a numerical range, it modifies that range by extending the boundaries above and below the numerical values set forth.

Numerical ranges recited herein by endpoints include all numbers and fractions subsumed within that range (e.g. 1 to 5 includes 1, 1.5, 2, 2.75, 3, 3.90, 4, and 5). It is also to be understood that all numbers and fractions thereof are presumed to be modified by the term “about.”

The formulations of the present invention can comprise, consist essentially of, or consist of, the essential as well as optional ingredients and components described herein. As used herein, “consisting essentially of” means that the formulation or component may include additional ingredients, but only if the additional ingredients do not materially alter the basic and novel characteristics of the claimed formulations or methods.

The term “topical application,” as used herein, means to apply or spread the formulations of the present invention onto the surface of mammalian skin, ocular tissue, or mucosa.

The phrase “topical” or “topically acceptable” as used herein, means that the formulations or components thereof so described are suitable for use in contact with human skin without undue toxicity incompatibility, instability, allergic response, and the like.

The phrase “effective amount” as used herein means an amount of a compound or formulation sufficient to significantly induce a positive benefit, preferably a positive skin appearance or feel benefit, including independently the benefits disclosed herein, but low enough to avoid serious side effects, i.e., to provide a reasonable benefit to risk ratio, within the scope of sound judgment of the skilled artisan.

The term “treating” includes prophylactic and/or therapeutic treatments. The term “prophylactic or therapeutic” treatment is art-recognized and includes administration to the subject of one or more of the subject compositions. If it is administered prior to clinical manifestation of the unwanted condition (e.g., disease or other unwanted state of the subject animal) then the treatment is prophylactic (i.e., it protects the host against developing the unwanted condition), whereas if it is administered after manifestation of the unwanted condition, the treatment is therapeutic (i.e., it is intended to diminish, ameliorate, or stabilize the existing unwanted condition or side effects thereof).

As used herein, a therapeutic that “prevents” a disorder or condition refers to a compound that, in a statistical sample, reduces the occurrence of the disorder or condition in the treated sample relative to an untreated control sample, or delays the onset or reduces the severity of one or more symptoms of the disorder or condition relative to the untreated control sample.

A “patient,” “subject,” or “individual” are used interchangeably and refer to either a human or a non-human animal. These terms include mammals, such as humans, primates, livestock animals (including bovines, porcines, etc.), companion animals (e.g., canines, felines, etc.) and rodents (e.g., mice and rats). In some embodiments, the subject is a human.

A “therapeutically effective amount” or a “therapeutically effective dose” of a drug or agent is an amount of a drug or an agent that, when administered to a subject will have the intended therapeutic effect. The full therapeutic effect does not necessarily occur by administration of one dose, and may occur only after administration of a series of doses. Thus, a therapeutically effective amount may be administered in one or more administrations. The precise effective amount needed for a subject will depend upon, for example, the subject's size, health and age, and the nature and extent of the condition being treated.

As used herein, “concurrent administration” refers to any form of administration of two or more different therapeutic agents such that the second agent is administered while the previously administered therapeutic agent is still effective in the body (e.g., the two agents are simultaneously effective in the patient, which may include synergistic effects of the two agents).

### Topical Formulations

In particular embodiments, the topical formulation comprises an omega-3 fatty acid component comprising eicosapentaenoic acid (EPA), docosahexaenoic acid (DHA), or a combination thereof; melatonin; vitamin D3; and at least one topical carrier. While not being bound by theory, it is believed that the formulations of the present disclosure provides in certain embodiments a triple action approach of 1) rebalancing dysbiosis, 2) strengthening surface barriers to prevent toxic substances, allergens, or pathogens from entering the body, and 3) reducing chronic inflammation. More particularly, it is believed that the ingredients in the present formulations, especially the omega-3 fatty acids, and melatonin, act surprisingly synergistically when applied topically, thereby 1) rebalancing dysbiosis, 2) strengthening surface barriers to prevent toxic substances, allergens, or pathogens from entering the body, and 3) reducing chronic inflammation.

Omega-3 fatty acids are polyunsaturated fatty acids (PUFAs) that are widely distributed in nature, being important cell constituents In particular, they play an important role in the human diet and in human physiology. The three main types of omega-3 fatty acids involved in human physiology are α-linolenic acid (ALA), found especially in plant oils, and eicosapentaenoic acid (EPA) and docosahexaenoic acid (DHA), both commonly found in marine oils.

In particular embodiments, the omega-3 fatty acid component comprises EPA and DHA. In some embodiments, the EPA and DHA have an EPA:DHA weight ratio ranging from about 0.5:1.5 to about 1.5:0.5. In other embodiments, the EPA:DHA weight ratio ranges from about 0.75:1.25 to about 1.25:0.75; about 0.8:1.2 to about 1.2:0.8; or about 0.9:1.1 to about 1.1:0.9, while in still other embodiments, the EPA:DHA weight ratio is about 1:1. In some embodiments, the omega-3 fatty acid component comprises EPA or the DHA alone. The omega-3 fatty acids may be bound to triglycerides, phospholipids, wax esters, fatty alcohols, ethyl esters, or be in free form acids. In some embodiments, the fatty acids are not microencapsulated, while in other embodiments they are microencapsulated. Various methods of microencapsulation are known to those skilled in the art. In some embodiments, the topical formulation comprises about 2% to about 25%, 2% to about 20%, about 8% to about 20%, or about 8% to about 15% by weight of the omega-3 fatty acid component. In other embodiments, the topical formulation comprises about 2%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, or about 25% by weight of the omega-3 fatty acid component.

Any suitable source of omega-3 fatty acid can be used in the invention, including, but not limited to vegetable oils, marine oils such as fish oils and fish liver oils, and algae. Synthetic sources of omega-3 fatty acids also may be used. Possible vegetable oil sources include olive oil, soybean oil, canola oil, high oleic sunflower seed oil, high oleic safflower oil, safflower oil, sunflower seed oil, flaxseed (linseed) oil, peanut oil, evening primrose oil, borage oil, and blackcurrant oil. Suitable marine oils include fish liver oil, fish body oil, calanus, or krill derived oil. Fish sources include salmon oil, cod/pollock/haddock liver oil, herring oil, mackerel oil, anchovy oil, anchovies, sardine oil, menhaden oil, tuna or shark liver oil. In some embodiments, the omega-3 fatty component comprises a purified and highly concentrated fish oil or EPA/DHA or DPA, and in other embodiments, the omega-3 fatty component comprises a natural, non-winterized cod liver oil.

In some embodiments, the omega-3 fatty acid source is a purified fish oil, such as a purified cod or other fish liver oil.

Omega-3 fatty acids, especially those sourced from marine oils, are readily susceptible to oxidation, contributing to a strong odor. Oxidation and a strong fish odor may affect their utility in a topical formulation. Encapsulation, such as microencapsulation, of marine oils is one approach to controlling oxidation and strong odors. Nevertheless, the present formulations are formulated such that the omega-3 fatty acids do not readily oxidize, without requiring encapsulation. Accordingly, in some embodiments, the omega-3 fatty acids are not microencapsulated.

Exemplary methods of producing omega-3 fatty acid compositions from a fish oil are described in U.S. Pat. Nos. 10,722,542, 10,245,247, and 9,827,218, each of which is specifically incorporated herein by reference.

Melatonin (N-acetyl-5-methoxytryptamine and its isomers) is a small ubiquitous molecule that acts as a hormone, anti-inflammatory and anti-oxidant in mammal cells. It is produced throughout the body and found in high concentrations in the gut, liver and bile where it is believed to protect the gastro-intestinal tract from being destroyed by the harsh digestive milieu. Melatonin is involved in lipid raft mobility and regulating cell membrane stiffness and tight junctions, in addition to being the 24 hour rhythm setter not only for human cells but also for the microbiome and it is also a strong sleep influencer. Similar to omega-3 fatty acids, it also has antimicrobial effects and thus serves as crucial microbiome balancer.

It is believed that melatonin also influence cellular cyto-skeleton and thereby influences attachment of tight junctions. Low melatonin content in cells and organ fluids are typically associated with chronic inflammation. It is further believed that melatonin is an omega-3 potentiator. Low omega-3 content in cells is by itself consistently associated to risk of chronic. Omega-3 fatty acids are also known as powerful anti-oxidants, anti-inflammatory agents and have antimicrobial activity on a long range of pathogens associated with dysbiosis. Melatonin also an effective anti-oxidant is may protect omega-3 stability and functionality.

Melatonin is also found in many plants, plankton and fish where it is believed to be important companion to lipids to protect them from being oxidized by free radicals. Cod and fish liver oils, in addition to being rich in omega-3 fatty acids, also contain significant amounts of melatonin and its natural isomers. This may explain why cod liver oil in traditional medicine (before the use of modern industrial production methods) was especially known for an anti-pain or arthritis effect since the melatonin family also has very potent anti-inflammation effects that work in conjunction with the omega-3 oils. While not being bound by theory, it is believed that that when melatonin is dissolved directly into omega-3 oil in some embodiments of the present disclosure, more potency is created compared to the separate applications of melatonin and omega-3 fatty acids. Again not being bound by theory, melatonin seems to need omega-3 to be fully activated (similar to cannabinoids).

In some embodiments, the present formulations comprise about 0.01% to about 15%, 0.01% to about 5%, about 0.01 to about 3%, about 0.01 to about 2%, about 0.01% to about 1%, about 0.1% to about 5%, about 0.1% to about 3%, about 0.1% to about 2%, about 0.1% to about 1%, about 0.5% to about 5%, about 0.5% to about 3%, about 0.5% to about 1.5%, or about 0.5% to about 1% by weight of melatonin. In other embodiments, the topical formulation comprises about 0.1%, about 0.2%, about 0.3%, about 0.4%, about 0.5%, about 0.6%, about 0.7%, about 0.75%, about 0.8%, about 0.9%, about 1%, about 1.5%, about 2%, about 2.5%, about 3%, about 4%, or about 5% by weight of the melatonin.

In some embodiments when the topical formulation is formulated for application to the skin, the topical formulation comprises up to about 5% of melatonin by weight of the formulation. In some embodiments, a topical formulation for the skin comprises about 0.01% to about 5%, about 0.01% to about 3%, about 0.01% to about 1%, or about 0.01% to about 0.5% by weight of the melatonin.

The present topical formulations further comprise vitamin D3 (cholecalciferol). Vitamin D (ergocalciferol-D2, cholecalciferol-D3, alfacalcidol) is a fat-soluble vitamin that helps your body absorb calcium and phosphorus. Vitamin D3 is also known for its anti-microbial actions as well as skin protector against UV radiation damage. In some embodiments, the topical formulation comprises about 0.005 to about 1%, about 0.01 to about 1%, about 0.01% to about 0.1%, or about 0.01% to about 0.02% by weight of the vitamin D3. In other embodiments, the topical formulation comprises about 0.01%, about 0.02%, about 0.03%, about 0.04, about 0.05%, about 0.06%, about 0.07%, about 0.08%, about 0.09%, about 0.10% by weight of the vitamin D3.

In some embodiments, the topical formulation further comprises β-glucan. β-glucans are a group of β-D-glucose polysaccharides naturally occurring in the cell walls of cereals, bacteria, and fungi, with significantly differing physicochemical properties dependent on source. Typically, β-glucans form a linear backbone with 1-3 β-glycosidic bonds but vary with respect to molecular mass, solubility, viscosity, branching structure, and gelation properties, causing diverse physiological effects in animals. They are sometimes used as medicine. β-glucans are taken by mouth for high cholesterol, diabetes, cancer, HIV/AIDS, high blood pressure, and canker sores. Oats contain large amounts of beta glucans which have anti-inflammatory effects. While not being bound by theory, it is believed that beta glucans have the ability to stabilize fatty acids, such as the omega-3 fatty acids used herein, and emulsify them in a water based formulation.

β-glucans form a natural component of the cell walls of bacteria, fungi, yeast, and cereals such as oat and barley. Each type of β-glucan comprises a different molecular backbone, level of branching, and molecular weight which affects its solubility and physiological impact. One of the most common sources of β(1,3) D-glucan for supplement use is derived from the cell wall of baker's yeast. β-glucans found in the cell walls of Saccharomyces cerevisiae contain a 1,3 carbon backbone with elongated 1,6 carbon branches. Other sources include seaweed, and various mushrooms, such as lingzhi, shiitake, chaga, and maitake.

In some embodiments, the present topical formulations comprise a plant-based β-glucan. In some embodiments, the plant based β-glucan is from oat fiber. Oat fiber β-glucans are also known for antimicrobial and anti-inflammatory actions.

While not being bound by theory, it is believed that the β-glucan, such as oat fiber β-glucan, may bind to, emulsify, or create a natural “microencapsulation” of omega-3 fatty acids. Thus, the β-glucan helps prevents separation of the omega-3 fatty acids from the other ingredients and thereby is useful for maintaining formulation stability. Oat fiber β-glucans are also known for antimicrobial and anti-inflammatory actions.

In some embodiments, the topical formulation comprises about 1% to about 10%, about 1 to about 5%, about 1 to about 3%, about 1 to about 2.5%, or about 2% to about 3% by weight of the β-glucan. In other embodiments, the topical formulation comprises about 1%, about 2%, about 2.5%, about 3%, about 4%, about 5%, about 6%, about 7%, about 8%, about 9%, or about 10% by weight of the β-glucan.

In some embodiments, the composition further comprises γ-linolenic acid (GLA). Suitable sources of GLA include, without limitation, borage oil. Accordingly, in some embodiments, the composition comprises borage oil. In some embodiments, the composition comprises about 1% to about 10%, about 1% to about 5%, about 1% to about 4%, about 1% to about 3% by weight of the borage oil. In other embodiments, the composition comprises about 1%, about 2$, about 3%, about 4%, or about 5% by weight of the borage oil.

In some embodiments, the topical formulation further comprises eucalyptus oil. Eucalyptus oil has a pleasant odor and additionally has wide-ranging benefits including decongestant, anti-inflammatory, antispasmodic, antiseptic, antifungal, antimicrobial, antiviral, and antibacterial properties. In some embodiments, the topical formulation comprises about 0.1 to about 5%, about 0.1 to about 3%, about 0.1 to about 2%, about 0.1 to about 1%, about 0.5 to about 2%, about 0.5 to about 1.5, or about 0.5 to about 1% by weight of eucalyptus oil.

In still further embodiments, the topical formulation comprises at least one cannabinoid. Cannabinoids are compounds found in cannabis. They are believed to modulate barrier embedded TRP receptor activity and work in synergy with omega-3 fatty acids. The most notable cannabinoid is the phytocannabinoid tetrahydrocannabinol (THC) (Delta9-THC or Delta8-THC), the primary psychoactive compound in cannabis. Cannabidiol (CBD) is another major constituent of the plant. There are at least 144 different cannabinoids isolated from cannabis, exhibiting varied effects.

Synthetic cannabinoids are manufactured artificially. They encompass a variety of distinct chemical classes: the classical cannabinoids structurally related to THC, the nonclassical cannabinoids (cannabimimetics) including the aminoalkylindoles, 1,5-diarylpyrazoles, quinolines, and arylsulfonamides as well as eicosanoids related to endocannabinoids.

Studies have shown that a CBD/CBDa combination had anti-inflammatory effects comparable to dexamethazone. Additionally, the present inventors found in cell studies that omega-3 fatty acids and melatonin by concomittant supplement use had synergistic actions on inflammation, microbiome regulation and surface barrier strengthening.

Accordingly, in some embodiments, the topical formulation comprises at least one cannabinoid chosen from cannabidiol (CBD), cannabidiolic acid (CBDA), cannabinol (CBN), Cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), and any combination thereof.

The amounts of cannabinoids may vary depending on the particular mode of application. For example, for formulations intended for use on the skin, the amount of the cannabinoids may be up to about 15% by weight of the formulation, while formulations for application to the mucosa may contain cannabinoids in an amount of up to about 5%. In some embodiments, the topical formulation comprises about 0.1% to about 15% by weight of the cannabinoid. In some embodiments, the topical formulation comprises about 0.1 to about 10%, about 0.1 to about 5%, or about 0.1% to about 1% by weight of the at least one cannabinoid. In still other embodiments, the topical formulation comprises about. 0.1%, about 0.2%, about 0.3%, about 0.4%, about 0.5%, about 1%, about 2%, about 3%, about 4%, about 5%, about 6%, about 7%, about 8%, about 9%, about 10%, about 11%, about 12%, about 13%, about 14%, or about 15% by weight of the at least one cannabinoid.

In some embodiments, the topical formulation further comprises ferulic acid. Ferulic acid is derived from plant extracts and has natural antimicrobial, anti-oxidant, anti-inflammatory effects, thereby preventing or reducing omega-3 fatty acid oxidation. Ferulic acid has been tested for favorable action on tight junctions. Finally, ferulic acid is used as natural food preservative.

In some embodiments, the topical formulation comprises about 0.01% to about 5%, about 0.01-3%, or about 0.1 to 1% by weight of the ferulic acid. In other embodiments, the topical formulation comprises about 0.1%, about 0.2%, about 0.3%, about 0.4%, about 0.5%, about 0.6%, about 0.7%, about 0.8%, about 0.9%, about 1.0% by weight of the ferulic acid.

Additional vitamins may be included in the present topical formulations. In some embodiments, the topical formulation further comprises vitamin A, vitamin E, or both.

In one embodiment, the topical formulation comprises vitamin A (e.g., retinyl palmitate). In some embodiments, the topical formulation comprises about 0.01% to about 5%, about 0.01% to about 1.5%, or about 0.1% to about 1% by weight of vitamin A. In particular embodiments, the formulation comprises about 0.01%, about 0.02%, about 0.03%, about 0.04%, about 0.05%, about 0.06%, about 0.07%, about 0.08%, about 0.09%, about 0.1%, about 0.2%, about 0.3%, about 0.4%, about 0.5%, about 0.6%, about 0.7%, about 0.8%, about 0.9%, or about 1% by weight of vitamin A.

In some embodiments, the composition does not comprise, or is substantially free from vitamin A.

In still further embodiments, the topical formulation comprises a source of medium chain fatty acids. Medium-chain fatty acids (MCFAs) are saturated or unsaturated fatty acids found at high concentrations in food such as coconut oil, with lauric acid (C12:0) representing ˜50% of its fat content, and palm kernel oil. In some embodiments, the source of MCFAs comprises coconut oil.

In some embodiments, the topical formulation comprises about 1% to about 10%, about 1% to about 5%, or about 2% to about 3% by weight of the coconut oil. In other embodiments, the topical formulation comprises about 1%, about 2%, about 3%, about 4%, about 5%, about 6%, about 7%, about 8%, about 9%, or about 10% by weight of the coconut oil.

The topical formulation may, in some embodiments, be formulated as a gel, lotion, solution, cream, ointment, oil, dressing, foam, or film. In particular embodiments, the topical formulation is formulated as a cream, ointment, or lotion. Numerous topical carriers are known in the art and are useful for formulating the present topical formulations. The viscosity of the topical formulation can advantageously have variable degrees of viscosity by simply changing lipid:water content ratio, and thus be adjusted to particular applications.

The carrier may contain one or more topically acceptable solid, semi-solid or liquid fillers, diluents, solvents, extenders and the like. The carrier may be solid, semi-solid or liquid. The carrier can be in some embodiments substantially liquid. The carrier can itself be inert or it can possess dermatological benefits of its own. Concentrations of the carrier can vary with the carrier selected and the intended concentrations of the components.

Suitable carriers include conventional or otherwise known carriers that are topically acceptable. The carrier should also be physically and chemically compatible with the essential components described herein, and should not unduly impair stability, efficacy or other use benefits associated with the formulations of the present invention. Preferred components of the formulations of this invention should be capable of being comingled in a manner such that there is no interaction which would substantially reduce the efficacy of the formulation under ordinary use situations.

The type of carrier utilized herein depends on the type of product form desired for the formulation. The topical formulations useful in the subject invention may be made into a wide variety of product forms such as are known in the art. Carriers can contain a topically acceptable diluent. As used herein, “diluent” includes materials in which the material can be dispersed, dissolved, or otherwise incorporated, such as a lipophilic diluent/carrier. Solutions according to the subject invention typically include a topically acceptable diluent. Solutions useful in the subject invention can contain from about 60% to about 99.99% of the diluent.

Aerosols according to the subject invention can be formed by adding a propellant to a solution such as described above. Exemplary propellants include chloro-fluorinated lower molecular weight hydrocarbons, Additional propellants that are useful herein are described in Sagarin, Cosmetics Science and Technology, 2nd Edition, Vol. 2, pp. 443-465 (1972), incorporated herein by reference. Aerosols are typically applied to as a spray-on product.

Carriers can comprise an emulsion comprising a hydrophilic phase comprising a hydrophilic component, e.g., water or other hydrophilic diluent, and a hydrophobic phase comprising a hydrophobic component, e.g., a lipid, oil or oily material. As well known to one skilled in the art, the hydrophilic phase will be dispersed in the hydrophobic phase, or vice versa, to form respectively hydrophilic or hydrophobic dispersed and continuous phases, depending on the formulation ingredients. In emulsion technology, the term “dispersed phase” is a term well-known to one skilled in the art which means that the phase exists as small particles or droplets that are suspended in and surrounded by a continuous phase. The dispersed phase is also known as the internal or discontinuous phase. The emulsion may be or comprise (e.g., in a triple or other multi-phase emulsion) an oil-in-water emulsion or a water-in-oil emulsion such as a water-in-silicone emulsion. Oil-in-water emulsions typically comprise from about 1% to about 50% of the dispersed hydrophobic phase and from about 1% to about 98% of the continuous hydrophilic phase; water-in-oil emulsions typically comprise from about 1% to about 98% of the dispersed hydrophilic phase and from about 1% to about 50% of the continuous hydrophobic phase. The emulsion may also comprise a gel network, such as described in “Application of Emulsion Stability Theories to Mobile and Semisolid Oil-in-Water Emulsions”, Cosmetics and Toiletries, vol. 101, November 1986, pp. 73-92, which is incorporated by reference herein. Preferred emulsions are further described below.

The topical formulations of the subject invention, including but not limited to lotions, oils and creams, may comprise a topically acceptable emollient. Such formulations can contain from about 2% to about 50% of the emollient. Emollients tend to lubricate the skin, increase the smoothness and suppleness of the skin, prevent or relieve dryness of the skin, and/or protect the skin. Emollients are typically water-immiscible, oily or waxy materials. A wide variety of suitable emollients are known and may be used herein. Sagarin, Cosmetics, Science and Technology 2nd Edition, Vol. 1, pp. 32-43 (1972).sub.; incorporated herein by reference, contains numerous examples of materials suitable as an emollient. Lotions and creams according to the present invention generally comprise a solution carrier system and one or more emollients. Lotions typically comprise from about 1% to about 20%, preferably from about 5% to about 10%, of emollient; from about 50% to about 90%, preferably from about 60% to about 80%, water. A cream typically comprises from about 5% to about 50%, preferably from about 10% to about 20%, of emollient; and from about 45% to about 85%, preferably from about 50% to about 75%, water.

Ointments of the present invention may comprise a simple carrier base of animal or vegetable oils or semi-solid hydrocarbons (oleaginous); absorption ointment bases which absorb water to form emulsions; or water soluble carriers, e.g., a water soluble solution carrier. Ointments may further comprise a thickening agent, such as described in Sagarin, Cosmetics, Science and Technology. 2nd Edition, Vol. 1, pp. 72-73 (1972), incorporated herein by reference, and/or an emollient. For example, an ointment may comprise from about 2% to about 10% of an emollient; and from about 0.1% to about 2% of a thickening agent.

Formulations of this invention useful for cleansing (“cleansers”) are formulated with a suitable carrier, e.g., as described above, and preferably contain one or more topically acceptable surfactants in an amount which is safe and effective for cleansing. For example, such formulations contain from about 1% to about 90%, more preferably from about 5% to about 10%, of a topically acceptable surfactant. The surfactant is can be selected from anionic, cationic, nonionic, zwitterionic, amphoteric and ampholytic surfactants, as well as mixtures of these surfactants. Such surfactants are well known to those skilled in the detergency art. Nonlimiting examples of possible surfactants include isoceteth-20, sodium methyl cocoyl taurate, sodium methyl oleoyl taurate, sodium lauryl sulfate, and betaines such as described herein. See U.S. Pat. No. 4,800,197, to Kowez et al., issued Jan. 24, 1989, which is incorporated herein by reference in its entirety, for exemplary surfactants useful herein. Examples of a broad variety of additional surfactants useful herein are described in Mccutcheon's Detergents and Emulsifiers, North American Edition (1986), published by Allured Publishing Corporation, which is incorporated herein by reference in its entirety. The cleansing formulations can optionally contain, at their art-established levels, other materials which are conventionally used in cleansing formulations.

The physical form of the cleansing formulations can be, for example, formulated as bars, liquids, gels, hair conditioners, hair tonics, pastes, or mousses. Bars are most preferred since this is the form of cleansing agent most commonly used to wash the skin. Preferred rinse-off cleansing formulations, such as shampoos, include a delivery system adequate to deposit sufficient levels of actives on the skin and scalp. A preferred delivery system involves the use of insoluble complexes. For a more complete disclosure of such delivery systems, see U.S. Pat. No. 4,835,148, Barford et al., issued May 30, 1989, incorporated herein by reference in its entirety.

The formulation may also take the form of a cosmetic formulation that may be applied to mammalian skin, ocular tissues, and mucosa. In some embodiments the formulation is designed for application to the eyelids. For example, some non-limiting examples of forms include solutions, suspensions, lotions, oils, creams, gels, toners, sticks, pencils, ointments, pastes, foams, powders, mousses, shaving creams, wipes, strips, patches, electrically-powered patches, wound dressing and adhesive bandages, hydrogels, film-forming products, facial and skin masks, cosmetics, and the like.

In some embodiments, the topical carrier comprises water, hyaluronic acid, lecithin, glycerin, dimethylsulfoxide (DMSO), xanthan, or a combination thereof. Additional carriers include alcohol, oils (such as mineral oil), liquid petrolatum, white petrolatum, glycerin, polyethylene glycol and propylene glycol. Penetration enhancers, emulsifying agents, suspending agents, colorants, fragrances may also be incorporated into the formulation.

In more particular embodiments, the topical carrier comprises water, hyaluronic acid, lecithin, glycerin, dimethylsulfoxide (DMSO), and/or xanthan.

In some embodiments, the composition comprises about 0.1 to about 2% by weight of DMSO. In some embodiments, the composition comprises about 0.1%, about 2%, about 0.3%, about 0.4%, about 0.5%, about 0.6%, about 0.7%, about 0.8%, about 0.9%, about 1%, about 11%, about 1.2%, about 1.3%, about 1.4%, about 1.5%, about 1.6%, about 1.7%, about 1.8%, about 1.9%, or about 2% by weight of the DMSO.

In some embodiments, the topical formulation comprises about 0.1% to about 5%, about 0.1% to about 3%, about 0.1% to about 2%, about 0.1% to about 1.5%, about 0.5% to about 5%, about 0.5% to about 3%, about 0.5% to about 2%, or about 0.5% to about 1.5% by weight of the hyaluronic acid. In other embodiments, the topical formulation comprises about 0.5%, about 0.6%, about 0.7%, about 0.8%, about 0.9%, about 1%, about 1.1%, about 1.2%, about 1.3%, about 1.4%, about 1.5%, about 1.6%, about 1.7%, about 1.8%, about 1.9%, about 2%, about 2.5%, about 3%, about 3.5%, about 4%, about 4.5%, or about 5% by weigh of hyaluronic acid.

In some embodiments, the formulation comprises about 0.05% to about 5% by weight of xanthan. In other embodiments, the formulation comprises about 0.1 to about 1% by weight of xanthan.

In some embodiments, the composition comprises about 0.5% to about 10% by weight of lecithin. In other embodiments, the composition comprises about 1% to about 5% by weight of lecithin.

In some embodiments, the composition comprises about 0.05% to about 10% by weight of glycerin. In other embodiments, the composition comprises about 1% to about 10%, about 2% to about 10% or about 2% to about 5% by weight of glycerin.

In some embodiments, the topical formulation comprises about 60% to about 85%, about 60% to about 85%, about 65% to about 80%, about 70% to about 80%, about 60% to about 75%, about 65% to about 75%, or about 65% to about 70% by weight of water. In other embodiments, the topical formulation comprises about 60%, about 61%, about 62%, about 63%, about 64%, about 65%, about 66%, about 67%, about 68%, about 69%, about 70%, about 71%, about 72%, about 73%, about 74%, about 75%, about 76%, about 77%, about 78%, about 79%, 80%, about 81%, about 82%, about 83%, about 84%, or about 85% by weight of the water.

In some embodiments, the topical formulation of any one of claims is formulated for application to skin. In other embodiments, the topical formulation is formulated for application to mucosa. In other embodiments, the topical formulation is formulated for application to the ocular tissue or applied to instruments as lubricant or anti-microbial agent or bio-film inhibitor.

### Additional Bioactive Agents

In some embodiments, the topical formulation further comprises at least one additional biologically active agent (typically Rx or OTC drugs). The topical formulation may act as a drug delivery system for a variety of biologically active agents. Classes of biologically active compounds include, without limitation, antibiotics, analgesics, anti-inflammatory agents, antihistamines, anti-cancer agents, immunosuppressants, TRP modulators, enzyme inhibitors, anti-convulsants, hormones, muscle relaxants, anti-spasmodics, ophthalmic agents, prostaglandins, anti-depressants, anti-psychotic substances, hair growth agents, trophic factors, and vaccines.

Antibiotics include without limitation aminoglycosides (e.g., gentamicin, tobramycin, netilmicin, streptomycin, amikacin, neomycin), bacitracin, corbapenems (e.g., imipenem/cislastatin), cephalosporins, colistin, methenamine, monobactams (e.g., aztreonam), penicillins (e.g., penicillin G, penicillin V, methicillin, natcillin, oxacillin, cloxacillin, dicloxacillin, ampicillin, amoxicillin, carbenicillin, ticarcillin, piperacillin, mezlocillin, azlocillin), polymyxin B, quinolones, and vancomycin; and bacteriostatic agents such as chloramphenicol, clindanyan, macrolides (e.g., erythromycin, azithromycin, clarithromycin), lincomyan, nitrofurantoin, sulfonamides, tetracyclines (e.g., tetracycline, doxycycline, minocycline, demeclocyline), and trimethoprim. Also included are metronidazole, fluoroquinolones, and ritampin.

Antihistamines include pyrilamine, chlorpheniramine, tetrahydrazoline, acrivastine, azelastine, bilastine, bromodiphenhydramine, brompheniramine, buclizine, carbinoxamine, cetirizine, chlorodiphenhydramine, chlorpheniramine, clemastine, cyclizine, cyproheptadine, desloratadine, dexbrompheniramine, dexchlorpheniramine, dimenhydrinate, dimetindene, diphenhydramine, doxylamine, ebastine, embramine, fexofenadine, hydroxyzine, levocabastine, levocetirizine, loratadine, meclizine, mirtazapine, olopatadine, orphenadrine, phenindamine, pheniramine, phenyltoloxamine, promethazine, pyrilamine, quetiapine, qupatadine, trazodone, tripelennamine, triprolidine, cimetidine, famotidine, lafutidine, lizatidine, ranitidine, roxatidine tiotidine, clobenpropit, ABT-239, ciproxifan, conessine, A-349,821, thioperamide.

Anti-inflammatory agents include corticosteroids, nonsteroidal anti-inflammatory drugs (e.g., aspirin, phenylbutazone, indomethacin, sulindac, tolmetin, ibuprofen, piroxicam, and fenamates), acetaminophen, phenacetin, gold salts, chloroquine, D-Penicillamine, methotrexate colchicine, allopurinol, probenecid, and sulfinpyrazone.

Topical steroids include alclometasone, hydrocortisone, diflorasone, triamcinolone, clobetasol, Clobetasol propionate, clocortolone, flurandrenolide, amcinonide, desonide, mometasone, fluocinonide, halcinonide, halobetasol, desoximetasone, methylprednisolone aceponate, betamethasone valerate, betamethasone dipropionate, and amcinonide.

Muscle relaxants include mephenesin, methocarbomal, cyclobenzaprine hydrochloride, trihexylphenidyl hydrochloride, levodopa/carbidopa, and biperiden.

Anti-spasmodics include atropine, scopolamine, oxyphenonium, and papaverine.

Analgesics include aspirin, phenybutazone, idomethacin, sulindac, tolmetic, ibuprofen, piroxicam, fenamates, acetaminophen, phenacetin, morphine sulfate, codeine sulfate, meperidine, nalorphine, opioids (e.g., codeine sulfate, fentanyl citrate, hydrocodone bitartrate, loperamide, morphine sulfate, noscapine, norcodeine, normorphine, thebaine, nor-binaltorphimine, buprenorphine, chlornaltrexamine, funaltrexamione, nalbuphine, nalorphine, naloxone, naloxonazine, naltrexone, and naltrindole), procaine, lidocain, tetracaine and dibucaine.

Ophthalmic agents include sodium fluorescein, rose bengal, methacholine, adrenaline, cocaine, atropine, alpha-chymotrypsin, hyaluronidase, betaxalol, pilocarpine, timolol, timolol salts, and combinations thereof.

Anti-cancer agents include alkylating agents, platinum agents, antimetabolites, topoisomerase inhibitors, antitumor antibiotics, antimitotic agents, aromatase inhibitors, thymidylate synthase inhibitors, DNA antagonists, farnesyltransferase inhibitors, pump inhibitors, histone acetyltransferase inhibitors, metalloproteinase inhibitors, ribonucleoside reductase inhibitors, TNF alpha agonists/antagonists, endothelin A receptor antagonists, retinoic acid receptor agonists, immuno-modulators, hormonal and antihormonal agents, photodynamic agents, and tyrosine kinase inhibitors.

Prostaglandins are art recognized and are a class of naturally occurring chemically related, long-chain hydroxy fatty acids that have a variety of biological effects.

Anti-depressants are substances capable of preventing or relieving depression. Examples of anti-depressants include imipramine, amitriptyline, nortriptyline, protriptyline, desipramine, amoxapine, doxepin, maprotiline, tranylcypromine, phenelzine, and isocarboxazide.

Hormones include estrogens (e.g., estradiol, estrone, estriol, diethylstibestrol, quinestrol, chlorotrianisene, ethinyl estradiol, mestranol), anti-estrogens (e.g., clomiphene, tamoxifen), progestins (e.g., medroxyprogesterone, norethindrone, hydroxyprogesterone, norgestrel), antiprogestin (mifepristone), androgens (e.g., testosterone cypionate, fluoxymesterone, danazol, testolactone), anti-androgens (e.g., cyproterone acetate, flutamide), thyroid hormones (e.g., triiodothyronne, thyroxine, propylthiouracil, methimazole, and iodixode), and pituitary hormones (e.g., corticotropin, sumutotropin, oxytocin, and vasopressin). Hormones are commonly employed in hormone replacement therapy and/or for purposes of birth control. Steroid hormones, such as prednisone, are also used as immunosuppressants and anti-inflammatoires.

Enzyme inhibitors are substances which inhibit an enzymatic reaction. Examples of enzyme inhibitors include edrophonium chloride, N-methylphysostigmine, neostigmine bromide, physostigmine sulfate, tacrine, tacrine, 1-hydroxy maleate, iodotubercidin, p-bromotetramisole, 10-(alpha-diethylaminopropionyl)-phenothiazine hydrochloride, calmidazolium chloride, hemicholinium-3, 3,5-dinitrocatechol, diacylglycerol kinase inhibitor I, diacylglycerol kinase inhibitor II, 3-phenylpropargylamine, N6-monomethyl-L-arginine acetate, carbidopa, 3-hydroxybenzylhydrazine, hydralazine, clorgyline, deprenyl, hydroxylamine, iproniazid phosphate, 6-MeO-tetrahydro-9H-pyrido-indole, nialamide, pargyline, quinacrine, semicarbazide, tranylcypromine, N,N-diethylaminoethyl-2,2-diphenylvalerate hydrochloride, 3-isobutyl-1-methylxanthne, papaverine, indomethacind, 2-cyclooctyl-2-hydroxyethylamine hydrochloride, 2,3-dichloro-a-methylbenzylamine (DCMB), 8,9-dichloro-2,3,4,5-tetrahydro-1H-2-benzazepine hydrochloride, p-aminoglutethimide, p-aminoglutethimide tartrate, 3-iodotyrosine, alpha-methyltyrosine, acetazolamide, dichlorphenamide, 6-hydroxy-2-benzothiazolesulfonamide, and allopurinol.

Additional topical biological agents include anthralin, coal tar, calcipotrieneammonium lactate/urea, aloe vera, resveratrol, resorcinol, acitretin, calcipotriene, salicylic acid, tazarotene, benzocaine, iodoquinol; class: topical anti-infectives, borage oil, evening primrose, eucalyptus oil, doxepin, adapalene, alefacept, aminolevulinic acid, amoxicillin, clavulanic acidazelaic acid benzoyl peroxide, capsaicin, cephalexin, chloroxine, ciclopirox, clindamycin, clioquinol, clotrimazole, collagenase clostridium histolyticum, doxycycline, erythromycin, etanercept, etretinate, famciclovir, fluocinolone, fluorouracil, fusidic acid, imiquimod, isoniazid, itraconazole, ketoconazole, lidocaine, lindane, meclocycline, metronidazole, minoxidil, mometasone, mupirocin, pimecrolimus, pramoxine, sodium sulfacetamide, tacrolimus, tazarotene, tea tree oil, terbinafine, zinc oxide.

The present topical formulations may be advantageously provided in any convenient package including airless “cosmetic” pumps, bottles, squeeze tubes, vials, ampules, syringes, and the like. The topical formulation may be provided as individual dosage forms or otherwise marked to provide a measured dose as needed.

### Methods of Administration and Treatment

The present formulations may be applied topically to the skin, mucosa, ocular tissue and/or eye lids to treat or prevent a variety of disorders. In some embodiments, the application is dermal or transdermal. The formulations may be applied to any area of the skin or mucosa in need of thereof. In some embodiments, the formulation is applied to the nasal mucosa or eye lids.

The effective amount of the formulation may vary according to the condition being treated and site of treatment. In some embodiments, the effective amount comprises about 25 to about 1000 mg, about 25 to about 500 mg, about 25 to about 200 mg, about 25 to about 150 mg, about 50 to about 100 mg, about 50 to about 900 mg, about 50 to about 800 mg, about 50 to about 700 mg, about 50 to about 600 mg, about 50 to 500 mg, about 50 to about 400 mg, about 50 to about 300 mg, about 50 to about 200 mg, or about 50 to about 150 mg. to about 10 mL, about 0.1 to about 9 mL, about 0.1 to about 8 mL, about 0.1 to about 7 mL, about 0.1 to about 6 mL, about 0.1 to about 5 mL, about 0.1 to about 4 mL, about 0.1 to about 3 mL, about 0.1 to about 2 mL, about 0.1 to about 1 mL, about 0.3 to about 10 mL, about 0.3 to about 9 mL, about 0.3 to about 8 mL, about 0.3 to about 7 mL, about 0.3 to about 6 mL, about 0.3 to about 5 mL, about 0.3 to about 4 mL, about 0.3 to about 3 mL, about 0.3 to about 2 mL, about 0.3 to about 1 mL, about 0.5 to about 10 mL, about 0.5 to about 9 mL, about 0.5 to about 8 mL, about 0.5 to about 7 mL, about 0.5 to about 6 mL, about 0.5 to about 5 mL, about 0.5 to about 4 mL, about 0.5 to about 3 mL, about 0.5 to about 2 mL, about 0.5 to about 1 mL, about 1 to about 10 mL, about 1 to about 9 mL, about 1 to about 8 mL, about 1 to about 7 mL, about 1 to about 6 mL, about 1 to about 5 mL, about 1 to about 4 mL, about 1 to about 3 mL, or about 1 to about 2 mL. In particular embodiments, the effective amount comprises about 50 mg to about 200 mg of the formulation.

In one embodiment, a method for treating or preventing dysbiosis or barrier “leaks” of the skin, ocular tissue, or mucosa of a subject in need thereof comprises applying to the skin or mucosa an effective amount of a topical formulation described herein. In some embodiments, the dysbiosis or barrier is on the skin, while in other embodiments, the location is the mucosa. The mucosa can be nasal, oral, genital, or anal. In some embodiments, the mucosa is nasal mucosa.

Accordingly, the present disclosure provides a method for treating or preventing a mucosal site condition in a subject in need thereof comprising application the topical formulation described herein to the mucosa of the subject.

The nose, more specifically the nasal mucosa, is one of the body's defense systems to fight air-born microbes, allergens, and toxins. It is unique in its way of cleaning and warming the air and removing larger particles. The abundant mucosal layer covering the surfaces further prevent pathogens from entering the cells, and its rich vascularization enables quick absorption of medications.

In some embodiments, the mucosal site condition is pollen allergy, chronic rhinosinusitis, nasal dysbiosis, asthma (such as exercise induced asthma), viral infection, bacterial infection, or any combination thereof.

S. aureus is present is in the nasal cavity, and can be a source of hospital infections, particularly post-surgery. Accordingly, the topical formulation can be applied to the nasal mucosa before and after a surgical or other hospital procedure.

The topical formulation may be applied to the nasal mucosa in any convenient manner, such as by using a finger, a nasal spray, a swab, or squeeze tube. The topical formulation can be applied to the nasal mucosa at least once every 2 to 3 days. In some embodiments, the topical formulation is applied to the mucosa 2, 3, or 4 times a day. In other embodiments, the topical formulation is applied on an as-needed basis. In some embodiments, about 50 mg to about 200 mg or about 150 mg of the topical formulation is applied to each nostril.

In some embodiments, the compositions of present invention can be delivered using a small needle-free nasal spray device, which can allow (self) administration with little or no prior training to deliver a desired dose. The apparatus can comprise a reservoir containing a quantity of the composition. The apparatus may comprise a pump spray for delivering one or more metered doses to the nasal cavity of a subject. The device may advantageously be single dose use or multi-dose use. It further may be designed to administer the intended dose with multiple sprays, e.g., two sprays, e.g., one in each nostril, or as a single spray, e.g., in one nostril, or to vary the dose in accordance with the body weight or maturity of the patient. In some embodiments, nasal drops may be prepacked in pouches or ampoules that may be opened immediate prior to use and squeezed or squirted into the nasal passages.

The nose is also connected to the eye through tear ducts and to the brain through direct nerves. Chronic nose tissue inflammation is typically found in people with dry eyes, sleep disorders, pollen allergy and of course rhinosinusitis. Chronic nose inflammation also leads to increased susceptibility to viral infections. Furthermore, there is a strong correlation between nasal, oral cavity and intestinal dysbiosis.

Thus, the present invention uses takes advantage of nasal application to fight chronic inflammation that may not be perceived as related to the nose per se such, as CNS disorders.

Since poor sleep quality is common feature of chronic inflammation, the present inventors predicted that the topical formulation would have a positive effect on sleep when applied to the nasal mucosa or to the eyelids in patients with dry eye symptoms. Accordingly, a method for promoting sleep in a subject in need thereof comprises applying the topical formulation described herein to the nasal mucosa or eyelids of the subject. The topical formulation is advantageously applied to the eyelids and/or each nostril in the evening, for example about 0.5 to about 2 hours before the subject's bedtime.

Nasal application also can, in some embodiments, reduce inflammation in the nasal mucosa, thereby improving breathing. This also may contribute to improved sleep.

Circadian disruption is the hallmark of jet lag and international traveling. Long distance air travel is also associated with increased risk of viral infections presumably by dried nasal mucosa. Applying the formulation in the nose before, under and after travel may serve both as prevention of viral infections and promote sleep adaptation to new time zones.

In some embodiments, application of the formulation to the eyelids also treats or prevents sleeping disorders. In some embodiments, the formulation is applied to the just the eyelids, while in other embodiments, the formulation is applied to the eyelids and the nose to promote sleep.

In some embodiments, the mucosa is the oral mucosa. In some embodiments, the mucosal site condition is gingivitis, periodontitis, oral lichen planus, herpes labialis, candidiasis, ulcer, pruritus or any combination thereof. The topical formulation may be applied in any convenient manner, such as by using a finger, a spray, a swab, squeeze tube, or toothbrush. The topical formulation can be applied to the oral mucosa at least once a day. In some embodiments, the topical formulation is applied to the mucosa 2, 3, or 4 times a day. In other embodiments, the topical formulation is applied on an as-needed basis.

One particular application is to use the formulation as a lubricant and anti-dysbiosis material to intubation tubes, catheters, vaginal specula, or a wound dressing for surgical incisions or for stomie.

In some embodiments, the method provides treatment of or reducing the symptoms of a dermatological condition comprising administering to a patient in need thereof an effective amount of a formulation described herein to a site of the dermatological condition on the subject.

UV radiation is today considered not only a cancerogenic factor but also a prime driver for skin aging through fatty acid oxidation, disruption of skin barrier and dysbiosis and chronic inflammation. The result is often premature wrinkles, skin dryness, discoloration, keratosis, and skin cancers. The formulation is believed to possess UV radiation protection by reducing dermal inflammation when applied to the skin surface.

Psoriasis is a skin disease, often marked by red scaly patches kin build up into bumpy red patches covered with white scales. They can grow anywhere, but most appear on the scalp, elbows, knees, and lower back. There are several different types of psoriasis. In most cases, people have one type at a time. Sometimes symptoms go away. Then, another type of psoriasis crops up in response to a trigger. Types of psoriasis included herein include but are not limited to plaque-type psoriasis, guttate psoriasis, pustular psoriasis, inverse psoriasis, erythrodermic psoriasis, nail psoriasis, psoriasis of the scalp, and psoriatic arthritis.

Eczema, also called dermatitis, is not one specific skin condition. Several types of eczema exist, and sometimes a person develops more than one type. Eczema includes but is not limited to atopic dermatitis, contact dermatitis, dyshidrotic dermatitis, hand dermatitis, neurodermatitis, nummular dermatitis, occupational dermatitis, seborrheic dermatitis and stasis dermatitis. Other common dermatological conditions include rash, hives, contact dermatitis, insect bite, allergic reaction, rosacea, acne, cold sores, blisters, hives, actinic keratosis, fungal infections, insect stings, burns, frost-bite, sun burn post-surgical cuts, abrasions, basal cell carcinoma, squamous cell carcinoma, melanoma, bed sores, parasitic infection, spider bites, shingles, infected wounds or any combination thereof. Eczema also may be caused by or exacerbated by frequent hand washing. Acne-like dermatitis as a result of mask wearing (such as for virus prevention or in medical settings) also may be treated or prevented by applying the present compositions to the skin in the area of the mask.

Cancer patients undergoing radiation therapy frequently endure skin conditions, ranging from minor itching and irritation to peeling and burns. In the some embodiments, radiation therapy damage may be treated or prevented by applying the present formulations to the damage prone area of skin.

The treatment of dermatological conditions comprises applying to the skin of a subject in need thereof an effective amount of a topical formulation described herein. The application may be at least once a day. In some embodiments, the topical formulation is applied to the 2, 3, or 4 times a day. In other embodiments, the topical formulation is applied on an as-needed basis. After applying the topical formulation, the site of application may be bandaged or wrapped if desired.

The present disclosure further provides a method for the treatment or prevention of disorders of the eye, including dry eye, ocular tension (glaucoma), or macular degeneration. The topical formulation may be applied to the eyelids, nasal mucosa, the ocular mucosa, or all. The application to the eye may be may be achieved by eyedropper, airless pump, squeeze tube or syringe. In some embodiments, the formulation is applied directly to the eye surface.

A method of treating or preventing dry eye comprises in some embodiments applying a topical formulation to the eyelids and/or nasal mucosa in a subject in need thereof with or without applying it in the nasal mucosa. The formulation can be applied every other day, once a day, or 2, 3, or 4 times a day. In some embodiments, the topical formulation is applied once a day or several times a day when treatment is initiated, and the dosing frequency is subsequently reduced once symptoms improve. Alternatively, the topical formulation can be applied on an as-needed basis, such as when symptoms of dry eye appear. In an alternative embodiment, a topical formulation as described herein may be applied directly to the ocular tissue to treat dry eye. The ocular application may be achieved by eye dropper, airless “cosmetic” pump, squeeze tube or syringe.

For chronic eye conditions, delivery of active agents to the eye while avoiding or reducing systemic exposure to the active agents is desirable. It has been found that topical eyelid skin application exhibited a localized drug absorption and specific drug accumulation in the ocular tissues (Gerard Lee Lo See, Journal of Pharmaceutical Sciences, April 2019). Accordingly, in some embodiments, topical application onto the skin of the eyelid is advantageous, rather than the use of eye drops, and may improve patient compliance since eye drops frequently cause eye irritation or blurring. Another advantage is that eye lid application will not interfere with use of eye drops and can easily be combined with eye drop therapies if so desired. It is believed that one mode of action is through modulation of epithelial TRP receptors, inducing a brain activation that stimulate tear production, increase lubrication and reduce pain sensation. It is interesting to note that several periocular transcutaneous electrical stimulation studies find increase in tear flow and tear quality after stimulation in the same way as the inventor noticed after periocular application of the eye cream. (“Effectiveness of transcutaneous electrical stimulation combined with artificial tears for the treatment of dry eye: A randomized controlled trial” Ming-Ming Cai 1, Jie Zhang 2 Exp Ther Med. 2020 December; 20 (6): 175. doi: 10.3892/etm.2020.9305. Epub 2020 Oct. 9. This further strengthens the theory that the different ingredients in the formulation/invention chemically stimulate the same nerves/TRP receptors as electrical impulses and underlines the importance of finding ingredients that work in synergy.

The present topical formulations also may be used in a method for treating ear aches, such as those arising from ear infections. The method comprises applying a topical formulation described herein to the ear canal of a subject in need thereof. The application may be via ear drops, syringe or squeeze tube.

Formulations of the present invention are also useful for vaginal application. Vaginal application may treat or prevent vaginal dryness, yeast infections, vaginal or vulvar atrophy, or menopausal symptoms such as hot flashes.

### EXAMPLES

### Example 1

An exemplary topical formulation is set forth in Table 1:

TABLE 1

| Ingredient | Weight (g) | Weight % |
|---|---|---|
| Omega-3 | 30 | 13.5 (10) |
| Coconut oil | 5.8 | 2.61 |
| CBD | 0.65 | 0.29 |
| β-glucan | 5.7 | 2.56 |
| melatonin | 0.75 (nose) | 0.34 |
| | 1.0 (skin) | 0.45 |
| Ferulic acid | 1.0 (0.9) | 0.45 |
| | | 0.41 |
| xanthan | 0.86 | 0.39 |
| Vitamin D3 | 0.04 (liquid D3 1 | 0.018 |
| | mg) | |
| DMSO | 2.5 | 1.13 |
| water | 150 | 68 |
| glycerin | 16.6 | 7.5 |
| Lecithin powder | 5.6 | 2.5 |
| Hyaluronic acid | 2.4 | 1.08 |
| (bovine) | | |
| Eucalyptus oil | 1.65 | 0.8 |
| Total | 223.05 | |

The formulation of table 1 may be modified for the desired application, for example by adjusting the water content and by adjusting the amounts of the other ingredients as described throughout the specification.

In vitro cell studies demonstrated that the formulation has no cytotoxicity. Lab analysis also indicated antimicrobial and anti-biofilm effects

### Example 2: User Experience

During product development different formulations and ratios were tested on medical conditions thought to implicate epithelial barrier dysfunctions, dysbiosis and chronic inflammation. Our hypothesis was that attacking the immune response simultaneously at these 3 levels we would have “universally” effective formulations for a multitude of common medical ailments.

Many of the test subjects presented a multitude of conditions like dry eyes and glaucoma, sleep disturbances, sinusitis, eczema, psoriasis, chronic wounds, burns, actinic keratosis, dry mouth, skin photo aging with wrinkles and hyper pigmentation

Approximately 50 people tested the formulation of Table 1 to evaluate compliance, dosage, effectiveness, response time, and medical indications. Application of the formulation several times per day in the beginning of treatment, and reduction to once a day application was effective for most indications. Dosing in the nose was approximately 50 mg to 150 mg in each nostril. Dosing on the skin was approximately 50 mg to 150 mg for an area about the size of a user's forehead. The formulation typically dried within two minutes of being applied to the skin, and was well tolerated even when applied to the nostrils and the eyelid. The formulations also did not exhibit unpleasant or strong odors after application.

Application to the nostrils resulted in nasal airway opening within about 30 minutes. 5 people with chronic yellow coloring of nasal mucus noticed clearing and better nasal air flow.

Application to the nostrils and the eyelid reduced eye dryness within about 30 minutes. Out of 30 people with dry eyes, everybody reported less use of eye drops and less eye irritation, except 2 participants who felt the peri-orbital application of the cream exaggerated the problems and they stopped after a few days.

7 out of 9 optometrist-examined patients had 40% reduction in ocular surface disease index (OSDI) score after 6 weeks and recommended to continue use.

Three subjects wore contact lenses and also had dry eye. These subjects reported less use of eye drops and longer contact lens wear time without irritation.

Three subjects had both glaucoma and dry eye, and all responded with significant intraocular pressure reduction in addition to dry eye improvement.

Only a part of the participants had sleep issues but those who had reported less waking up during the night and less day time fatigue and registered deeper sleep per digital registration. Application to the nostrils induced sleep within 30 minutes

Three subjects with sleep apnea applied the cream in the nose at bed time, and registered fewer frequencies according to continuous digital recording.

Four subjects with facial actinic keratosis applying the cream 2 times per day experienced disappearance of the skin disorder after 3 to 5 weeks.

Five subjects with chronic eczema or seborrheic dermatosis obtained significant improvement after 1 week and 2 applications per day.

One subject was treated in hospital for 2 symmetrical post radiation chronic wounds, one wound was treated with conventional therapy the other side with the cream. After 10 days, the hospital decided to stop the convention treatment and encouraged the patient to continue with the more effective cream treatment.

A number of test people also tried the formulation to reduce craw feet and hyper pigmentation spots on wrists and face. Significant effects were noticed after 3 to 4 weeks for pigmentation and 2 to 3 months for craw feet.

Application to insect bites resulted in an anti-inflammatory effect (including reduced itching and swelling) was noted within about 15 minutes.

Three subjects with hyper sensitivity to insect bites applied the cream quickly after wasp or red ants stings and did not develop pain, redness, itch or swelling.

The formulation was also tried on vaginal dryness, swimmers ear, herpes simplex, distal neuralgia, all of which had positive response.

Application to the ear reduced ear ache within 20 minutes.

No side effects were noted except if peri-orbital use came too close to the eye ball (burning sensation) or using large amounts or too deep insertion into the nose creating sneeze reflex and burning.

Most test users spontaneously reported they enjoyed the smell of the cream and how fast it was absorbed into the skin without leaving any residue. Users further reported that they did not observe a fishy smell.

Although there have been described particular embodiments of the present invention of a new and useful topical formulations, it is not intended that such references be construed as limitations upon the scope of this invention. While the making and using of various embodiments of the present invention are discussed in detail above, it should be appreciated that the present invention provides many applicable inventive concepts that are embodied in a wide variety of specific contexts. Those of ordinary skill in the art will recognize numerous equivalents to the specific formulations and methods described herein. Such equivalents are considered to be within the scope of this disclosure and are covered by the claims.

### Example 3

| Ingredient | Weight (g) | Weight % |
|---|---|---|
| Omega-3 | 80 | 6.2 |
| Borage oil | 40 | 3.1 |
| Coconut oil | 36 | 2.8 |
| CBD oil | 7.5 | 0.58 |
| β-glucan | 22 | 1.7 |
| melatonin | 1.3 | 0.1 |
| Ferulic acid | 4 | 0.31 |
| xanthan | 3.4 | 0.26 |
| Vitamin D3 | 0.16 (liquid D3 1 | 0.96 |
| | mg) | |
| DMSO | 12.5 | 1.13 |
| water | 975 | 75% |
| glycerin | 68 | 5.2 |
| Lecithin powder | 28 | 2.2 |
| Hyaluronic acid | 12.5 | 1.13 |
| (bovine) | | |
| Retinyl | 3.4 | 0.07 |
| palmitate | | |
| Eucalyptus oil | 6 | 0.5 |
| Total | 1300 g | |

### Example 4

A skin cream was prepared according to the following formula. All ingredients are shown in weight (grams)

| Oil base (17%) | | Water base (83%) | |
|---|---|---|---|
| Omega Cure (1:1 EPA:DHA | 80.0 | Water | 975.0 |
| Coconut oil | 53.0 | Glycerin | 70.0 |
| Borage oil | 40.0 | Lecithin | 28.0 |
| Beta Glucan | 22. | Hyaluronic acid, | 12.5 |
| CBD 40% (Diluted raw | 6.5 (6.3-6.5) | Mogrosides | 5.0 |
| CBD resin (77%) with | | | |
| coco oil 1/1 ratio) | | | |
| Eucalyptus oil globulus | 6.0 (6.0-6.2) | Licorice root | 5.0 |
| Ferulic Acid | 6.2 | Total water phase= | 1085.5 g |
| 38. Xanthan | 3.4 | | |
| 9 Melatonin | 1.0 (0.75-1.5) | | |
| 10. Vitamin D3 | 0.160 | | |
| (using liquid vit | | | |
| D3 1 Mill/gram) | | | |
| DMSO | 6.5 (add into oil phase | | |
| | last, right before | | |
| | mixing with water phase) | | |

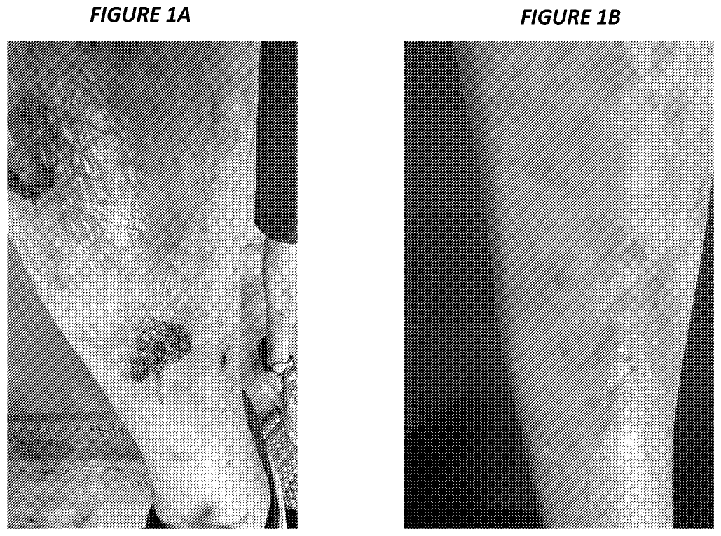

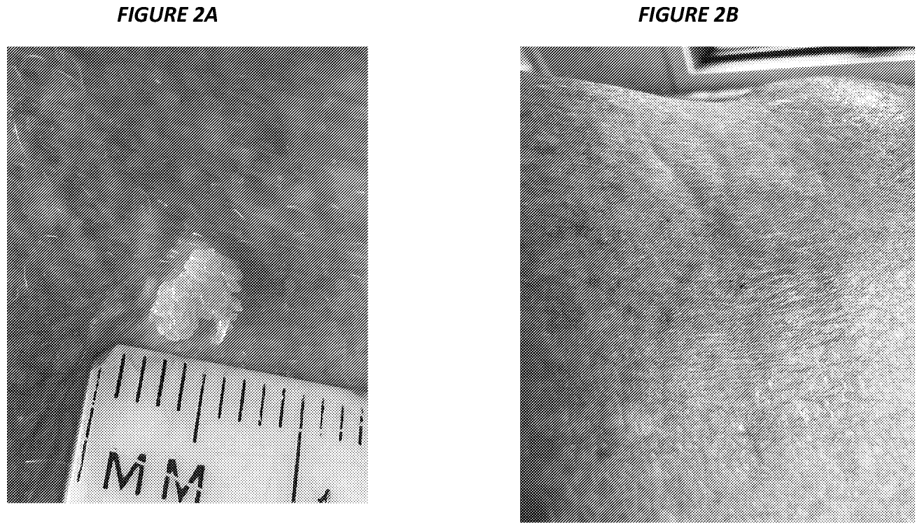

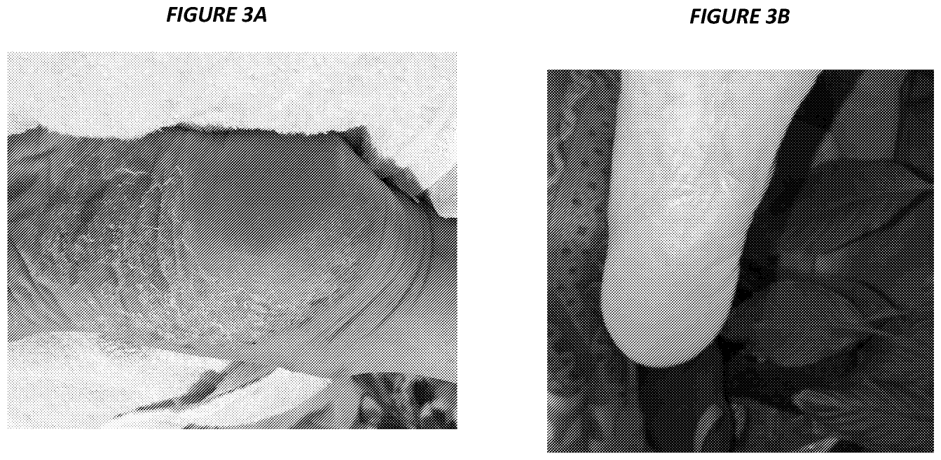

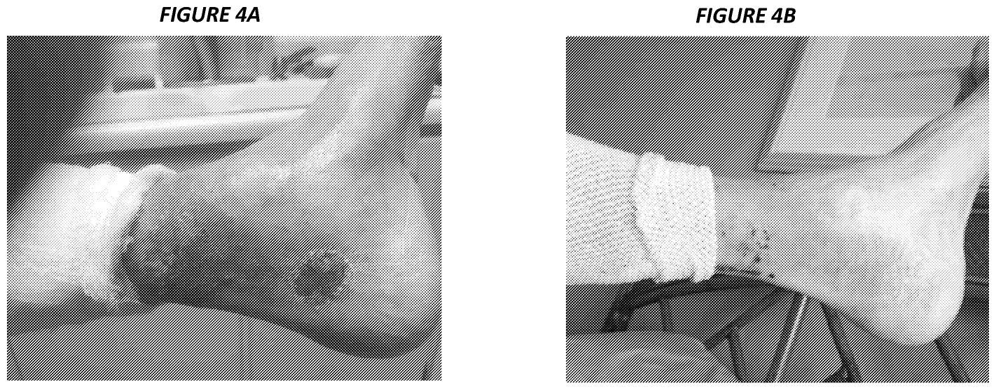

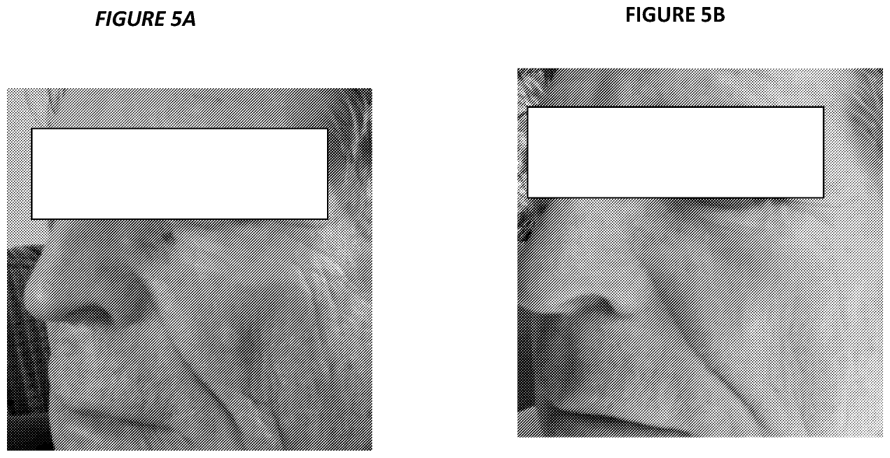

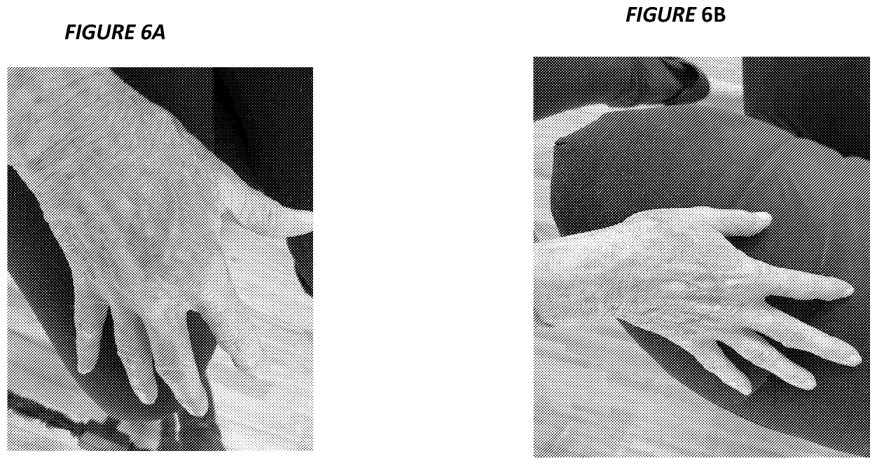

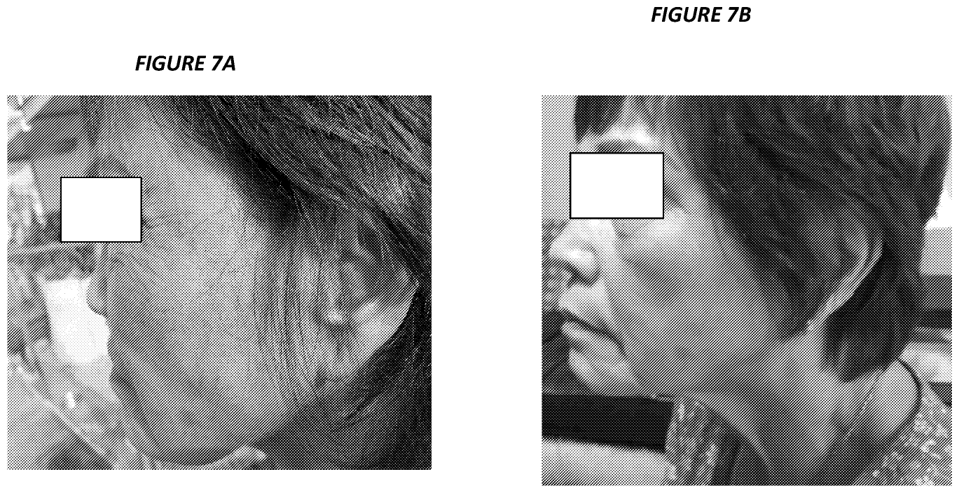

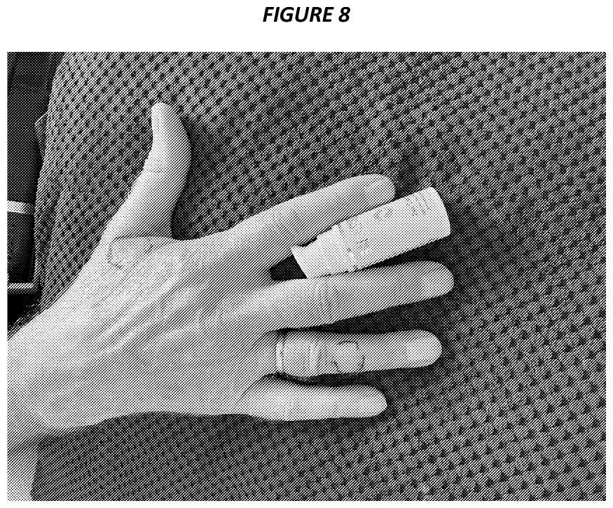

Customer experiences across a wide range of indications were evaluated. FIGS. 1A to 8 are photographic images demonstrating subject experiences with this composition. Specifically, subjects applied the crème formulation to the skin to successfully treat and or reduce the symptoms of chronic wound, papilloma, eczema or the foot, cancer irradiation burn, actinic keratosis, eczema of the hand, periorbital wrinkles, and wasp stings. Chronic wound treatment showed significant healing after three weeks of daily application. A papilloma was significantly diminished after 10 days of application of the crème. Chronic eczema in the foots was not visible and itching diminished after 2 weeks of treatment. A cancer irradiation burn was significantly healed after two and half weeks of application to the bur actinic keratosis was no longer visible after five weeks of treatment. Chronic eczema was no longer visible and itching diminished after two and half weeks of treatment. Periorbital wrinkles were reduced after 6 weeks of daily application. A wasp-allergic individual applied the crème after being stung twice. After 15 minutes, the subject experienced no pain, itching or redness at the site of the stings.

### INCORPORATION BY REFERENCE

All publications and patents mentioned herein are hereby incorporated by reference in their entirety as if each individual publication or patent was specifically and individually indicated to be incorporated by reference. In case of conflict, the present application, including any definitions herein, will control.

### EQUIVALENTS

While specific embodiments of the subject invention have been discussed, the above specification is illustrative and not restrictive. Many variations of the invention will become apparent to those skilled in the art upon review of this specification and the claims below. The full scope of the invention should be determined by reference to the claims, along with their full scope of equivalents, and the specification, along with such variations.

1. A topical formulation comprising:
an omega-3 fatty acid component comprising eicosapentaenoic acid (EPA), docosahexaenoic acid (DHA), or a combination thereof;
melatonin;
vitamin D3;
β-glucan; and
at least one topical carrier.

2. The topical formulation of claim 1, wherein the omega-3 fatty acid comprises EPA and DHA.

3. The topical formulation of claim 2, wherein the EPA and DHA have a EPA:DHA weight ratio ranging from about 0.5:1.5 to about 1.5:0.5.

4. The topical formulation of claim 3, wherein the EPA:DHA weight ratio ranges from about 0.75:1.25 to about 1.25:0.75; about 0.8:1.2 to about 1.2:0.8; or about 0.9:1.1 to about 1.1:0.9.

5. The topical formulation of claim 3, wherein the EPA:DHA weight ratio is about 1:1.

6. The topical formulation of any one of claims 1 to 5, wherein the omega-3 fatty acid consists essentially of the EPA and the DHA.

7. The topical formulation of any one of claims 1 to 6, wherein the omega-3 fatty source is a purified fish oil.

8. The topical formulation of claim 7, wherein the purified fish oil is a purified fish liver oil.

9. The topical formulation of any one of claims 1 to 8, wherein the topical formulation comprises:
about 2 to about 25% by weight of the omega-3 fatty acid component,
about 0.01 to about 1% by weight of the melatonin, about
about 0.005 to about 1% by weight of the vitamin D3, and
about 1% to about 10% by weight of β-glucan.

10. The topical formulation of claim 9, wherein the topical formulation comprises:
about 5 to about 15% by weight of the omega-3 fatty acid component,
about 0.01 to about 0.5% by weight of the melatonin, about
about 0.01 to about 0.1% by weight of the vitamin D3, and
about 1% to about 5% by weight of β-glucan.

11. The topical formulation of any one of claims 1 to 10, further comprising a source of γ-linolenic acid (GLA).

12. The topical formulation of claim 11, wherein the source of GLA is borage oil.

13. The topical formulation of claim 11 or 12, wherein the topical formulation comprises about 1 to about 10% by weight of the borage oil.

14. The topical formulation of claim 13, wherein the topical formulation comprises about 1 to about 5% by weight of the borage oil.

15. The topical formulation of any one of claims 1 to 14, further comprising eucalyptus oil.

16. The topical formulation of claim 15, wherein the topical formulation comprises about 0.1 to about 5% by weight of the eucalyptus oil.

17. The topical formulation of claim 16, wherein the topical formulation comprises about 0.1 to about 1% by weight of the eucalyptus oil.

18. The topical formulation of any one of claims 1 to 17, further comprising at least one cannabinoid.

19. The topical formulation of claim 18, wherein the cannabinoid is chosen from cannabidiol (CBD), cannabidiolic acid (CBDA), cannabinol (CBN), Cannabinolic acid (CBNA), cannabigerol (CBG), cannabigerolic acid (CBGA), cannabichromene (CBC), cannabichromenic acid (CBCA), cannabicyclol (CBL), cannabicyclolic acid (CBLA), cannabivarin (CBV), cannabivarinic acid (CBVA), tetrahydrocannabivarin (THCV), tetrahydrocannabivarinic acid (THCVA) cannabidivarin (CBDV), cannabidivarinic acid (CBDVA), cannabichromevarin (CBCV), cannabichromevarinic acid (CBCVA), cannabigerovarin (CBGV), cannabigerovarinic acid (CBGVA), and any combination thereof.

20. The topical formulation of claim 18 or 19, wherein the topical formulation comprises about 0.1 to about 15% by weight of the at least one cannabinoid.

21. The topical formulation of claim 20 wherein the topical formulation comprises about 0.1 to about 10%, about 0.1 to about 5%, about 0.1% to about 1% by weight of the at least one cannabinoid.

22. The topical formulation of any one of claims 1 to 21, further comprising ferulic acid.

23. The topical formulation of claim 22, wherein the topical formulation comprises about 0.1 to about 5% by weight of the ferulic acid.

24. The topical formulation of claim 23, wherein the topical formulation comprises about 0.1 to about 1% by weight of the ferulic acid.

25. The topical formulation of any one of claims 1 to 24, further comprising a vitamin A, vitamin E, or both.

26. The topical formulation of claim 25, wherein the formulation comprises the vitamin A.

27. The topical formulation of claim 26, wherein the topical formulation comprises about 0.01 to about 5% of the vitamin A

28. The topical formulation of claim 26, wherein the topical formulation comprises about 0.01 to about 2% of the vitamin A.

29. The composition of any one of claims 1 to 25, wherein the composition does not comprise vitamin A.

30. The topical formulation of any one of claims 1 to 29, further comprising a source of a medium chain fatty acid.

31. The topical formulation of claim 30, wherein the source of the medium chain fatty acid comprises coconut oil.

32. The topical formulation of claim 31, wherein the topical formulation comprises about 1 to about 10% by weight of the coconut oil.

33. The topical formulation of claim 32, wherein the topical formulation comprises about 1 to about 5% by weight of the coconut oil.

34. The topical formulation of any one of claims 1 to 33, wherein the formulation is formulated as a gel, lotion, solution, cream, ointment, oil, dressing, foam, spray, or film.

35. The topical formulation of claim 34, wherein the topical formulation is formulated as a cream, ointment, or lotion.

36. The topical formulation of any one of claims 1 to 35, wherein the topical carrier further comprises water, hyaluronic acid, dimethylsulfoxide, lecithin, and/or glycerin.

37. The topical formulation of claim 36, wherein the topical formulation comprises about 60 to about 85% by weight of the water.

38. The topical formulation of claim 37, wherein the topical formulation comprises about 65 to about 80% by weight of the water.

39. The topical formulation of any one of claims 1 to 38, wherein topical carrier comprises about 0.1 to about % by weight of the hyaluronic acid.

40. The topical formulation of claim 39, wherein the topical formulation comprises about 0.1-2% by weight of the hyaluronic acid.

41. The topical formulation of any one of claims 1 to 40, further comprising morgosides, licorice root, or both.

42. The topical formulation claim 41, wherein the composition comprises about 0.1 to about 1% by weight of licorice root.

43. The topical formulation claim 41, about 0.1 to about 0.5% by weight of licorice root.

44. The topical formulation of any one of claims 40 to 43, wherein the composition comprises about 0.1 to about 1% of morgosides.

45. The topical formulation of any one of claims 40 to 43, wherein the composition comprises about 0.1 to about 0.5% of morgosides.

46. The topical formulation of any one of claims 1 to 45, wherein the topical formulation is formulated for application to skin.

47. The topical formulation of any one of claims 1 to 46, wherein the formulation is formulated for application to mucosa.

48. The topical formulation of any one of claims 1 to 47, wherein the formulation is formulated for application to the eye lids.

49. The topical formulation of any one of claims 1 to 48, wherein the topical formulation further comprises at least one additional biologically active agent.

50. The topical formulation of claim 49, wherein the at least one biologically active agent is chosen from an antibiotic, an anti-inflammatory, an anti-cancer, an enzyme inhibitor, a TRP modulator, a topical steroid, or an analgesic.

51. A method for treating or preventing dysbiosis and/or strengthening surface barriers of the skin or mucosa of a subject in need thereof comprising applying to the skin or mucosa an effective amount of a topical formulation according to any one of claims 1 to 50.

52. The method of claim 51, wherein the dysbiosis and/or surface barrier is on the skin.

53. The method of claim 52 wherein the dysbiosis and/or surface barrier is on the mucosa.

54. The method of claim 53, wherein the mucosa is nasal, oral, ocular, genital, anal, or rectal.

55. The method of claim 54, wherein the mucosa is the nasal mucosa or oral mucosa.

56. The method of claim 55, wherein the mucosa is nasal mucosa.

57. A method for promoting sleep or treating a sleep disorder in a subject in need thereof comprising applying the topical formulation of any one of claims 1 to 50 to the eye lids or nasal mucosa of the subject.

58. The method of claim 57, wherein the sleep disorder is sleep apnea, jet lag, or insomnia.

59. A method for treating or preventing a mucosal site condition in a subject in need thereof comprising applying an effective amount the topical formulation according to any one of claims 1 to 50 to the mucosa of the subject.

60. The method of claim 59, wherein the mucosa is nasal, oral, ocular, genital, anal, or rectal mucosa.

61. The method of claim 60, wherein the mucosa is nasal mucosa.

62. The method of claim 61, wherein the mucosal site condition is pollen allergy, chronic rhinosinusitis, nasal dysbiosis, asthma, viral infection, bacterial infection, or any combination thereof.

63. The method of any one of claim 61 or 62, wherein the method comprises applying the formulation by nasal spray, swab, or squeeze tube.

64. The method of claim 63 wherein the mucosa is the oral mucosa.

65. The method of claim 64, wherein the mucosal site condition is mucosal dryness, gingivitis, periodontitis, oral lichen planus, herpes labialis, candidiasis, ulcer, Sjogren's syndrome, or any combination thereof.

66. A method for treating or preventing a dermatological condition in a subject in need thereof comprising applying an effective amount of the formulation according to any one of claims 1 to 50 to the skin of the subject.

67. The method according to claim 66, wherein the dermatological condition comprises eczema, pruritus, psoriasis, rash, hives, contact dermatitis, insect bite, allergic reaction, rosacea, acne, cold sores, blisters, hives, actinic keratosis, fungal infections, insect stings, burns, frost-bite, sun burn, UV radiation protection, post-surgical cuts, stomal site irritation, radiation therapy skin burns, photo-aging, abrasions, basal cell carcinoma, squamous cell carcinoma, melanoma, bed sores, parasitic infection, spider bites, shingles, infected wounds, seborrheic dermatitis, contact dermatitis, or any combination thereof.

68. A method for preventing or reducing dry eye, ocular tension, glaucoma, Sjogren's syndrome, or retinal conditions in a subject in need thereof comprising applying a formulation of any one of claims 1 to 50 to the eye or eye lids of a subject in need thereof.

69. The method of claim 68, further comprising applying the formulation to the nasal mucosa.

70. A method of treating vaginal dryness, vaginal or vulvar atrophy, or hot flashes comprising applying to the skin or mucosa an effective amount of a topical formulation according to any one of claims 1 to 50 to the vaginal mucosa of a subject in need thereof.

71. The method of claim 70, wherein the disorder or condition is vaginal dryness, vaginal or vulvar atrophy.

72. A perioperative treatment method for cataract or lasik surgery comprising applying a formulation of any one of claims 1 to 50 to the eye or eye lids of a subject in need thereof.

73. A method of preventing or reducing ear ache in a subject in need thereof comprising applying the formulation of any one of claims 1 to 50 to the ear canal of a subject in need thereof.

74. A method of topically delivering a biologically active agent comprising applying an effective amount of a composition according to claim 49 or 50 to the skin or mucosa of a subject in need thereof.

75. The method of any one of claims 51 to 74, wherein the effective amount of the topical formulation is about 50 mg to about 200 mg.
